(19) United States
(12) Patent Application Publication
(10) Pub. No.: US 2020/0138890 A1
(43) Pub. Date: May 7, 2020

(54) PHARMACEUTICAL COMPOSITION FOR PREVENTION AND TREATMENT OF PROSTATIC HYPERPLASIA AND ERECTILE DYSFUNCTION CAUSED BY ANDROPAUSE COMPRISING EXTRACT OF LESPEDEZA CUNEATA AND TRIGONELLAE SEMEN

(71) Applicants: Kyoeng-Soo Lee, Seongnam-si Gyeonggi-do (KR); DUHANBIO CO., LTD., Seoul (KR)

(72) Inventors: Kyoeng-Soo Lee, Seongnam-si Gyeonggi-do (KR); Sung-Giu Jin, Seoul (KR)

(73) Assignees: DUHANBIO CO., LTD., Seoul (KR); Kyoeng-Soo Lee, Seongnam-si Gyeonggi-do (KR)

(21) Appl. No.: 15/549,776

(22) PCT Filed: Jun. 7, 2017
(86) PCT No.: PCT/KR2017/005898

§ 371 Date: Aug. 9, 2017

(30) Foreign Application Priority Data
Jun. 5, 2017 (KR) 10-2017-0069466

(51) Int. Cl. A61K 36/48; A61P 15/12

(52) U.S. Cl. CPC A61K 36/48; A61K 2236/333; A61P 15/12

(57) ABSTRACT

The present invention relates to a pharmaceutical composition for prevention or treatment of prostatic hyperplasia among Aging Males' Symptoms, comprising a Lespedeza cuneata extract and a Trigonellae semen extract, and a preparation method thereof characterized in that the complex composition produced by mixing the Lespedeza cuneata extract and the Trigonellae semen extract has a mixing ratio by weight of 1˜10:1˜9 of the Lespedeza cuneata extract and the Trigonellae semen extract, preferably, a mixing ratio by weight of 1:1˜4. The pharmaceutical preparation according to the present invention comprising the Lespedeza cuneata extract or complex composition of the Lespedeza cuneata extract and the Trigonellae semen extract have an effect on improvement of erectile function and for prevention or treatment of prostatic hyperplasia at the same time, among Aging Males' Symptoms, without any side effect.

### CROSS-REFERENCE TO RELATED APPLICATION

This application claims under 35 U.S.C. § 119(a) the benefit of Korean Patent Application No. filed on, the entire contents of which are incorporated herein by reference.

### BACKGROUND

### (a) Technical Field

The present invention relates to a pharmaceutical compositions for the prevention and treatment of prostatic hyperplasia among Aging Males' Symptoms, more specifically, to a pharmaceutical composition and a preparation method thereof for the improvement of erectile function and the prevention and treatment of prostatic hyperplasia, among Aging Males' Symptoms, comprising a Lespedeza cuneata extract or a mixture of the Lespedeza cuneata extract and a Trigonellae semen extract as an effective component.

### (b) Background Art

Aging Males' Symptoms, or as a more academic term, Testosterone Deficiency Syndrome (TDS) is a disease which makes men aged 50s or 60s languid even though this period of time in life is an important period of time of and also a period of time to work hard. This term is originated from referring to any combinations of symptoms of frequent fatigue, decreased sexual function, facial flushing, memory loss, depression and the like in men aged 50s by Werner in 1939.

Aging Males' Symptoms (TDS) recently, verbatim, refers to typical symptoms such as decreased sexual instinct and vitality, erectile dysfunction, decreased muscle mass and bone density, passive attitude or the like, and clinical and biochemical syndromes in which the concentration of testosterone in the blood is lowered, wherein these typical symptoms and clinical and biochemical syndromes are occurred by decrease of testosterone, a male hormone which is produced in the testicles, due to the gradual problem of testicular function with age, and are experienced by men as their age increases.

Erectile dysfunction among the Aging Males' Symptoms means, according to the definition of the National Institutes of Health, that “erections are not triggered or maintained to consistently perform sex life satisfactorily.” The result of various epidemiologic studies is known to show a high prevalence rate in men after 50 years old, and thus to have a significant negative affect on the quality of life. The prevalence rate of erectile dysfunction has been reported to vary depending on regions. According to Massachusetts Males Aging Study (MMAS) in United States of America, it was reported to experience erectile dysfunction in 52% of men aged 40-70 years even though there is a difference in degree. In addition, according to Korea's report (Daewoo Securities industry analyst research report, 2001), it was reported that 32% of adults aged 30 or more has erectile dysfunction. To date, several pathophysiological mechanisms which cause erectile dysfunction is known, but among these, vascular pathophysiology is known to play the most important role in the development of erectile dysfunction. It is known that about half of the erectile dysfunction occurring in the age group of 50 years or more are caused by diseases of the vascular system.

In relation to the treatment of erectile dysfunction, recently, many studies such as the development of sildenafil derivatives such as Viagra have been conducted. These sildenafil derivatives act as mechanism to inhibit the activity of cGMP hydrolase, PDE-5, in order to increase the cGMP concentration in the NO-cGMP pathway, thereby playing a role to maintain the erection of the penis by inhibiting the contraction of the penis. However, currently, various side effects such as myocardial infarction, heart failure, hypotension, cerebral infarction, facial flushing, and failing of eyesight have been reported, and thus a substitute substance for the safe treatment of the erectile dysfunction is desperately required.

Korean Patent Publication No. 10-2015-0125849 discloses the use of the extract of Lespedeza cuneata as a functional food for improving male sexual function, and the use of the pharmaceutical composition containing β-sitostero-6′-linolenoyl-3-O-β-D-glucopyranoside isolated from the above extract, as a therapeutic agent for erectile dysfunction.

Recently, according to the study on the Aging Males' Symptoms improvement of the inventors (Korean J. Food Sci. Technol. Vol. 47, No. 4, pp. 492-498 (2015)), the experimental result was that the complex extract of Lespedeza cuneata and Trigonellae semen is useful for relieving Aging Males' Symptoms, that is to say, after administration of it to rats, testosterone, a male hormone which is used as an indicator of Aging Males' Symptoms, was significantly increased; the concentration of SHBG (sex hormone binding globulin) which binds to testosterone and then decreases its function was decreased; and a measured result of the total number of sperms and the number of active sperms were excellent; and so on; and thus, the complex extract of Lespedeza cuneata and Trigonellae semen has been expected to be developed as health materials which can help improve Aging Males' Symptoms.

In addition, the inventors' prior application (Application No. 10-2015-0003398) discloses a composition for the prevention and treatment of Aging Males' Symptoms in which the complex extracts of Lespedeza cuneata and Trigonellae semen improve Aging Males' Symptoms such as decreased lethargy and vitality, decreased muscle strength, increased body fat, increased fatigue and stress through an increase in male hormone, an increase in muscular endurance and strength, a decrease in body fat, a decrease in lipid in blood, an increase in the number of sperms and an increase in sperm motility

Meanwhile, the inventors of the present invention found that the complex extract of Lespedeza cuneata and Trigonellae semen has an excellent effect in improving erectile dysfunction, which is one of Aging Males' Symptoms, and in treating prostatic hyperplasia by significantly increasing the male hormone, i.e., testosterone, while continuing to conduct the study on the improvement of the Aging Males' Symptoms of the complex extract of Lespedeza cuneata and Trigonellae semen, and thus the inventors got an idea to achieve the present invention.

Benign prostatic hyperplasia (BPH) is also one of the most commonly occurring diseases in adult men, is reported to be present in 50% or more of men over 50 years old and in 80% or more of men over 80 years old, and thus has the highest frequency of urinary tract disorders in men. When the prostate gland becomes abnormally enlarged and grows to an abnormal size of 40 to 400 g, bladder storage symptoms such as frequent urination urinating 8 times a day or more, nocturia, and urinary urgency which is a unbearable feeling of urination while feeling strong and sudden micturition desire, and symptoms of bladder outlet obstruction such as retarded miction which takes a long time to urinate when urinating, interrupted micturition which results a cut off of the flow of urine, and a phenomenon that requires power, etc. are occurred.

The cause of prostatic hyperplasia has not been clearly elucidated, but it is thought that the cause is due to the change of male hormone according to aging, and testosterone and dihydrotestosterone (DHT) among others are the most important relevant causes. That is to say, testosterone in the blood causes DHT to be synthesized by 5-alpha reductase present in the prostate tissue. Dihydrotestosterone makes cell division of stromal cells and epithelial cells of prostate gland more active than testosterone, thereby leading to the development of prostate gland hypertrophy.

Unlike testosterone, dihydrotestosterone does not decrease in amount with aging, leading to an increase in α-l-adrenergic receptors in prostate gland cells to maintain the balance of the endocrine system, and finally, prostate gland hypertrophy occurs by continuously stimulating other growth factors that induce the proliferation of the prostate gland.

In addition, the treatment agent of prostatic hyperplasia can be largely classified into two kinds of 5-alpha reductase inhibitor and α-l-adrenergic receptor inhibitor. The 5-alpha reductase inhibitor inhibits the conversion of testosterone to DHT and has the advantage of delaying the progression of the accompanying disease. However, the 5-alpha reductase inhibitor is difficult to achieve immediate effects, so it requires treatment for at least 2 months to 1 year until the disease is improved, and the 5-alpha reductase inhibitor has the side effects such as erectile dysfunction, hyposexuality, ejaculatory disturbance, and abnormal hypertrophy of the breasts of the male breast. The alpha 1-adrenergic receptor inhibitor has the effect of immediately relieving symptoms associated with urination by blocking the alpha 1-adrenergic receptor, which is highly expressed in the prostate gland and the bladder neck, and relaxing the smooth muscle of the prostate gland. However, about 15% of patients experience side effect of syncope, dizziness, or orthostatic hypotension, and the adverse effect in the cardiovascular system can be caused in the case of older patients

Due to the side effects of these existing medicines, there have been attempts to find a solution from natural materials. For example, Korean Patent Application No. 10-2016-7015585 discloses a composition and a method useful for treating lower urinary tract symptoms, benign prostatic hyperplasia and erectile dysfunction using cranberry.

The present inventors have confirmed through a clinical experiment that the complex composition containing the natural components, i.e., Lespedeza cuneata and Trigonellae semen extract is excellent in the treatment and also improvement of prostate hyperplasia as well as male erectile dysfunction, and then have completed the present invention.

### SUMMARY OF THE DISCLOSURE

An object of the present invention is to provide a novel pharmaceutical composition which is effective in the improvement of erectile function or the prevention and treatment of prostatic hyperplasia, among Aging Males' Symptoms, in order to solve the above problems.

In addition, it is an object of the present invention to provide a functional food composition for the improvement of erectile function or for the prevention and treatment of prostatic hyperplasia, among Aging Males' Symptoms, by using the Lespedeza cuneata extracts and Trigonellae semen extracts which are components derived from natural materials, have no toxicity, have stability to the human body, have no side effects or resistance, and therefore can be safely used.

To achieve the above object, the present invention is characterized by being a pharmaceutical composition for preventing and treating prostatic hyperplasia, which comprises as an effective component a Lespedeza cuneata extract, a Trigonella semen extract, or a complex composition produced by mixing the Lespedeza cuneata extract and the Trigonella semen extract.

The pharmaceutical composition for the prevention and treatment of prostatic hyperplasia containing the complex composition produced by mixing a Lespedeza cuneata extract and a Trigonellae semen extract as the effective component according to the present invention is characterized in that the mixing ratio of the Lespedeza cuneata extract and the Trigonellae semen extract is 1:1˜9 by weight ratio, and preferably the mixing ratio of the Lespedeza cuneata extract and the Trigonellae semen extract is 1:1˜4 by weight ratio.

The Lespedeza cuneata extract and the Trigonellae semen extract according to the present invention can be extracted by a known natural material extraction method wherein each material, before extraction, is collected, washed cleanly and then crushed.

The extraction solvent may preferably comprise at least one solvent selected from water, and C1-C6 organic solvent, and the C1-C6 organic solvent may be selected from the group consisting of C1-C6 alcohol, acetone, ether, benzene, chloroform, ethyl acetate, methylene chloride, hexane, cyclohexane and petroleum ether. More preferably, the extraction solvent may be water, C1 to C6 alcohol and a mixed solvent thereof, and the alcohol is preferably ethanol, and most preferably, 50 to 90% ethanol produced by mixing water and ethanol may be used.

The amount of the solvent is preferably used in the amount of 3˜15 times by volume based on the total weight of the extract targets of Lespedeza cuneata and Trigonellae semen and more preferably is used in the amount of 3˜10 times by volume

The extraction temperature in the present invention is not particularly limited, but may be preferably from 50° C. to 100° C. The extraction time of the extract of the present invention is not particularly limited, but may be 2 to 6 hours.

The extract of the present invention can be extracted by the known natural substance extraction methods. For example, extraction methods conventional in the art such as a method using an extraction device such as supercritical extraction, subcritical extraction, high temperature extraction, high pressure extraction, or ultrasonic extraction, or the methods using the adsorbent resin including XAD and HP-20 may be used and preferably the heat extraction or the reflux extraction method may be used. The extraction step in the present invention may be repeated one to seven times, preferably one to three times.

Also, after extraction, the concentration and sterilization step can be performed, and the extract after the concentration and sterilization step can be used as a liquid phase. In the present invention, it is preferable that the extract is pulverized through the drying process by spray drying or freeze-drying, and then used.

In addition, a fermentation process for the Lespedeza cuneata extract, the Trigonella semen extract, or the mixture of the Lespedeza cuneata extract and the Trigonella semen extract of the present invention can be further performed. The fermentation process for the extracts of the present invention can be performed by the known fermentation methods, and the fermentation strain is not particularly limited, but may be lactic acid bacteria, preferably Bifidobacterium longum, Lactobacillus acidophilus, Lactobacillus bulgaricus, Streptococcus thermophiles, Bifidobacterium infantis, Lactobacillus casei, Lactobacillus plantarum, or a mixed strain of these lactic acid bacteria. The condition of fermentation may vary depending on the type of strain used.

In addition, the extracts and the mixture of the extracts of the present invention can be prepared in the form of a composition produced by mixing them with active ingredients known to be effective in the prevention or improvement of diseases or symptoms caused by prostate gland hypertrophy.

The composition according to the present invention can be formulated and used in various forms of oral formulations such as powders, solutions, tablets, granules, capsules, suspensions, emulsions, syrups or pills, or sterilized injection forms, etc. by the conventional methods known in the art suitable for each purpose, and may be administered orally or by a variety of routes including intravenous, intraperitoneal, subcutaneous, rectal, and topical routes, etc. Examples of the suitable carriers, excipients and diluents which may be included in the compositions according to the invention may include lactose, dextrose, sucrose, sorbitol, mannitol, xylitol, erythritol, maltitol, starch, acacia rubber, alginate, gelatin, calcium phosphate, calcium silicate, cellulose, methylcellulose, amorphous cellulose, polyvinylpyrrolidone, water, methylhydroxybenzoate, propylhydroxybenzoate, talc, magnesium stearate, and mineral oil, etc. In addition, the composition according to the present invention may further include fillers, anti-coagulants, lubricants, humectants, fragrances, emulsifiers, preservatives, etc.

When the composition according to the present invention is prepared in the form of a solid preparation for oral administration, at least one excipient such as starch, calcium carbonate, sucrose, lactose, gelatin or the like is mixed and formulated. Also, in addition to simple excipients, lubricants such as magnesium stearate and talc may be used.

When the compositions according to the invention is prepared in the form of oral liquid preparations, in addition to commonly used simple diluents such as water and liquid paraffin, various excipients such as humectants, sweetening agents, perfumes, preservatives or the like may be included.

When the composition according to the present invention is prepared in the form of a preparation for parenteral administration, aqueous sterilized injectable solutions, non-aqueous solutions, suspending agents, emulsifying agents, freeze-dried preparations, or suppositories may be included. The non-aqueous solutions or the suspensions may be propylene glycol, polyethylene glycol, vegetable oils such as olive oil, injectable esters such as ethyl oleate, etc. Base materials for injection may include conventional additives such as isotonic agents, suspending agents, emulsifying agents, stabilizing agents or preservatives.

In addition, the extracts and the mixture of the extracts of the present invention can be prepared in the form of a composition produced by mixing them with active ingredients known to be effective in the prevention or improvement of diseases or symptoms caused by prostate gland hypertrophy.

In addition, the present invention may be prepared in the form of a functional food compositions by using Lespedeza cuneata extract and the Trigonellae semen extract, and also by mixing the extracts and the mixture of the extracts of the present invention together with active ingredients known to be effective in the improvement of erectile function or in the prevention or improvement of prostatic hyperplasia, among Aging Males' Symptoms.

The pharmaceutical composition containing the Lespedeza cuneata extract or the complex composition produced by mixing the Lespedeza cuneata extract and the Trigonellae semen extract of the present invention can prevent the occurrence of prostatic hyperplasia among Aging Males' Symptoms without side effects, and can provide effects of relief and treatment of symptoms.

In addition, the present invention can have an effect to provide a functional food composition for the improvement of erectile function and for the prevention and treatment of prostatic hyperplasia, among Aging Males' Symptoms, by using the Lespedeza cuneata extracts and Trigonellae semen extracts which are components derived from natural materials, have no toxicity, have stability to the human body, have no side effects or resistance, and therefore can be safely used.

### BRIEF DESCRIPTION OF THE DRAWINGS

FIG. 1 is a graph showing the effect on cGMP in cells of extracts of Examples 1 to 11 of the present invention.

FIG. 2 is a graph showing the effect on cGMP in cells of the extract of Example 9 of the present invention according to concentration.

FIG. 3 is a graph showing the activity of 5-alpha reductase II in prostate glands excised from SD rats, in relation to the extracts of Examples 1 to 11 of the present invention.

FIG. 4 is a graph showing the activity of 5-alpha reductase II in prostate glands excised from SD rats, in relation to the extract of Example 9 of the present invention according to concentration.

FIG. 5 is a graph showing the activity of 5-alpha reductase II in prostate glands of SD rats who induce prostate gland hypertrophy by testosterone, in relation to the extracts of Examples 6 to 9 of the present invention

FIG. 6 is a graph showing the scores of questionnaire survey result for Aging Males' Symptoms of the subjects administered with the extract of Example 9.

FIG. 7 is a graph showing the scores of questionnaire survey result for the international erectile function index of the subjects administered with the extract of Example 9 of the present invention

### DETAILED DESCRIPTION

The present invention relates to a pharmaceutical composition for the prevention and treatment of prostatic hyperplasia, which is characterized by being the pharmaceutical composition containing as an effective ingredient a Lespedeza cuneata extract or a complex composition produced by mixing the Lespedeza cuneata extract and the Trigonella semen extract.

Lespedeza cuneata as used herein is known that the scientific name is Lespedeza cuneata G. Don and the countries of origin are Korea, Japan, Taiwan, India and Australia, etc. and known that it is a pharmacological material with the efficiency to invigorate the liver and the kidney, aid the lung yin ( [symbol] ), eliminate the extravasated blood ( [symbol] ) and make the swelling subside, wherein the Lespedeza cuneata functions to protect the kidneys, help the liver and make asthma subside. Besides, it is known that Lespedeza cuneata has efficacies such as antitussive effect and abscess removal effect, etc. Lespedeza cuneata is known to be used to control diseases such as ganacratia ( [symbol] —Symptom of sperm flow without knowing himself and without having sexual intercourse), enuresis, symptoms of the white and thick urine, stomachache, symptoms of the swollen and sore hypogastrium, coughing and asthma, childhood anemia, ophthalmopathy, and malignant furuncle in the breast, etc. However, Lespedeza cuneata has not been used simultaneously for Erectile dysfunction and Prostatic hyperplasia

In addition, the scientific name of Trigonellae semen is also referred to as Trigonella foenum-graecum L. and Fenugreek. The countries of origin are India, China, Southeast Asia, Europe, Egypt, Sudan, USA, Lebanon, and Algeria, etc. and the seed of Trigonellae semen is generally known to be sun-dried and then used medicinally. Trigonellae semen has a warm property and has a non-toxic property, and thus have been used in Africa, the Middle East, India and elsewhere to treat bladder and kidney diseases. Trigonellae semen is also used to treat a person who sheds cold sweat or has abdomen cold. Compendium of Materia Medica (Ben Cao Gang Mu) stated that Trigonellae semen is used to treat a man with sore back, the cold knees and the clammy scrotum, defeat all the deficiency colds and numbness, treat symptoms of frequent urination and treat cold symptoms inside stomach. Recently, Trigonellae semen has been known to be effective in maintaining balance between blood sugar and insulin in the body and also to be effective in controlling body weight, but it has not yet been used simultaneously for Erectile dysfunction and for Prostatic hyperplasia.

The pharmaceutical composition comprising the complex composition produced by mixing the Lespedeza cuneata extract and the Trigonellae semen extract according to the present invention is characterized in that the mixing ratio of the Lespedeza cuneata extract and the Trigonellae semen extract is 1:1˜9 by weight ratio, and preferably the mixing ratio of the Lespedeza cuneata extract and the Trigonellae semen extract is 1:1˜4 by weight ratio.

The Lespedeza cuneata extract and the Trigonellae semen extract according to the present invention can be extracted by a known natural material extraction method wherein each material, before extraction, is collected, washed cleanly and then crushed.

The extraction solvent may preferably comprise at least one solvent selected from water, and C1-C6 organic solvent, and the C1-C6 organic solvent may be selected from the group consisting of C1-C6 alcohol, acetone, ether, benzene, chloroform, ethyl acetate, methylene chloride, hexane, cyclohexane and petroleum ether. More preferably, the extraction solvent may be water, C1 to C6 alcohol and a mixed solvent thereof, and the alcohol is preferably ethanol, and most preferably, 50 to 90% ethanol produced by mixing water and ethanol may be used.

The amount of the solvent is preferably used in the amount of 3˜15 times by volume based on the total weight of the extract targets of Lespedeza cuneata and Trigonellae semen and more preferably is used in the amount of 3˜10 times by volume

The extraction temperature in the present invention is not particularly limited, but may be preferably from 50° C. to 100° C. The extraction time of the extract of the present invention is not particularly limited, but may be 2 to 6 hours.

The extract of the present invention can be extracted by the known natural substance extraction methods. For example, extraction methods conventional in the art, such as the method using the extraction devices such as supercritical extraction, subcritical extraction, high temperature extraction, high pressure extraction, or ultrasonic extraction, or the method using the adsorbent resin including XAD and HP-20 may be used and preferably the heat extraction or the reflux extraction method may be used. The extraction step in the present invention may be repeated one to seven times, preferably one to three times.

Also, after extraction, the concentration and sterilization step can be performed, and the extract after the concentration and sterilization step can be used as a liquid phase. In the present invention, it is preferable that the extract is pulverized through the drying process by spray drying or freeze-drying, and then used.

In addition, a fermentation process for the Lespedeza cuneata extract, the Trigonella semen extract, or the mixture of the Lespedeza cuneata extract and the Trigonella semen extract of the present invention can be further performed. The fermentation process for the extracts of the present invention can be performed by the known fermentation methods, and the fermentation strain is not particularly limited, but may be lactic acid bacteria, preferably Bifidobacterium longum, Lactobacillus acidophilus, Lactobacillus bulgaricus, Streptococcus thermophiles, Bifidobacterium infantis, Lactobacillus casei, Lactobacillus plantarum, or a mixed strain of these lactic acid bacteria. The condition of fermentation may vary depending on the type of strain used.

In addition, the composition prepared by the present invention can be prepared by adding active ingredients known to be effective in the prevention or improvement of diseases or symptoms caused by prostate gland hypertrophy to the Lespedeza cuneata extract or the mixture of the Lespedeza cuneata extract and the Trigonella semen extract.

The composition according to the present invention increases the concentration of cGMP in the cells, suggesting that the composition of the present invention can increase the expression of NO-producing enzymes in the excitatory nervous system and the vascular system, or inhibit the expression of PDE-5, thereby promoting the relaxation of smooth muscle cells of the corpus cavernosum penis or inhibiting the contraction thereof.

Also, the composition of the present invention, as a result of the measurement results of DPPH free radical scavenging activity and SOD (superoxide dismutase) activity, showed a significant increase in DPPH and SOD according to the concentration. These results demonstrate that the composition of the present invention has an antioxidative effect of inhibiting oxidation by active oxygen in the body, and the inhibition of oxidation in the body can inhibit the induction of aging and thus improve the symptoms of male erectile dysfunction which is closely related to aging.

When the composition according to the present invention is orally administered, the concentration in the blood of testosterone, i.e., a male sex hormone is increased. The increase in plasma of testosterone indicates that the composition of the present invention not only improves male erectile dysfunction but also has an effect on male reproductive function.

The composition according to the present invention can be formulated and used in various forms of oral formulations such as powders, solutions, tablets, granules, capsules, suspensions, emulsions, syrups or pills, or sterilized injection forms, etc. by the conventional methods known in the art suitable for each purpose, and may be administered orally or by a variety of routes including intravenous, intraperitoneal, subcutaneous, rectal, and topical routes, and the like. Examples of suitable carriers, excipients and diluents which may be included in the compositions according to the invention include lactose, dextrose, sucrose, sorbitol, mannitol, xylitol, erythritol, maltitol, starch, acacia rubber, alginate, gelatin, calcium phosphate, calcium silicate, cellulose, methylcellulose, amorphous cellulose, polyvinylpyrrolidone, Water, methylhydroxybenzoate, propylhydroxybenzoate, talc, magnesium stearate, and mineral oil, etc. In addition, the composition according to the present invention further includes fillers, anti-coagulants, lubricants, humectants, fragrances, emulsifiers, or preservatives, etc.

When the composition according to the present invention is prepared in the form of a solid preparation for oral administration, at least one excipient such as starch, calcium carbonate, sucrose, lactose, gelatin or the like is mixed and formulated. Also, in addition to simple excipients, lubricants such as magnesium stearate or talc may be used.

When the compositions according to the invention is prepared in the form of oral liquid preparations, in addition to commonly used simple diluents such as water and liquid paraffin, various excipients such as humectants, sweetening agents, perfumes, preservatives or the like may be included.

When the composition according to the present invention is prepared in the form of a preparation for parenteral administration, aqueous sterilized injectable solutions, non-aqueous agents, suspending agents, emulsifying agents, freeze-dried preparations, or suppositories may be included. The non-aqueous agents and suspending agents may be propylene glycol, polyethylene glycol, vegetable oils such as olive oil, injectable esters such as ethyl oleate, etc. may be used. Base materials for injection may include conventional additives such as isotonic agents, suspending agents, emulsifying agents, stabilizing agents and preservatives.

In addition, the present invention may be prepared in the form of a functional food compositions by using Lespedeza cuneata extract and the Trigonellae semen extract, and also by mixing the extracts and the mixture of the extracts of the present invention together with active ingredients known to be effective in the improvement of erectile function or in the prevention or improvement of prostatic hyperplasia, among Aging Males' Symptoms.

Hereinafter, the present invention will be described in detail with reference to examples and experimental examples. However, the following examples and experimental examples are provided only for illustrating the present invention, and the scope of the present invention is not limited by the examples and the experimental examples.

### EXAMPLES 1 TO 11

Preparation of Mixture of Lespedeza Cuneata Extract and Trigonellae Semen Extract

100 g of Lespedeza cuneata were prepared and crushed to a size of 20 mesh using the grinder. To the crushed Lespedeza cuneata powder, 800 ml of 80% aqueous ethanol solution was added and extraction was carried out at a temperature of 60 to 80° C. for 3 hours. The extraction step was further repeated twice under the same conditions. The resulted ethanol extract was filtered through the filter, and the resulting filtrate was concentrated, followed by sterilized at 95° C. for 1 hour, and then completely dried using a spray dryer to prepare a powder.

Also, 100 g of Trigonellae semen were prepared and crushed to a size of 20 mesh using the grinder. To the crushed Trigonellae semen powder, 800 ml of 80% aqueous ethanol solution was added and extraction was carried out at a temperature of 60 to 80° C. for 3 hours. The extraction step was further repeated twice under the same conditions. The resulted ethanol extract was filtered through the filter, and the resulting filtrate was concentrated, followed by sterilized at 95° C. for 1 hour, and then completely dried using the spray dryer to prepare a powder.

The obtained powder of the Lespedeza cuneata extract and the obtained powder of the Trigonellae semen extract were mixed according to the composition ratios of Examples 1 to 11 as shown in the following Table 1 to prepare the compositions of the present invention. Example 1 is an example including only Lespedeza cuneata extract, and Example 11 is an example using only Trigonella semen extract.

TABLE 1
Mixing ratio of Lespedeza cuneata extract
and Trigonellae semen extract (unit: g)

| | Examples | | | | | | | | | | |
|---|---|---|---|---|---|---|---|---|---|---|---|
| Components | 1 | 2 | 3 | 4 | 5 | 6 | 7 | 8 | 9 | 10 | 11 |
| Lespedeza | 1 | 0.9 | 0.8 | 0.7 | 0.6 | 0.5 | 0.4 | 0.3 | 0.2 | 0.1 | — |
| cuneata | | | | | | | | | | | |
| extract | | | | | | | | | | | |
| Trigonellae | — | 0.1 | 0.2 | 0.3 | 0.4 | 0.5 | 0.6 | 0.7 | 0.8 | 0.9 | 1 |
| semen | | | | | | | | | | | |
| extract | | | | | | | | | | | |

The compositions of the present invention prepared through Examples 1-11 were tested for the efficacy of prevention and treatment of prostate hyperplasia, a Aging Males' Symptoms symptom, through the following Experimental Examples.

### Experimental Example 1: Cyclic GMP Evaluation in Cells

The number of smooth muscle cells was counted, and the cells were transferred to a 100 mm dish, inoculated, and then cultured at 70%, and then treated with the extract prepared in Example 1-11 for 24 hours. The medium and cells were separated, and the medium was used as a test sample for cGMP quantification. After preparing the standard material and the test sample, 200 μl of each of standard material and test sample were placed in tubes containing the culture medium respectively. To the Goat anti-rabbit IgG microplates, 100 μl of each of standard material and test sample were added respectively, and 50 μl of cGMP conjugate and 50 μl of cGMP antibody solution were added to each well and then reacted on the horizontal orbital microplate shaker at room temperature for 2 hours. After discarding the liquid in the plate and washing the plate with the washing solution, 200 μl of pNPP substrate was added and allowed to react for 1 hour, and 50 μl of stop solution was added, and then the absorbance (405 nm) was measured with an ELISA reader.

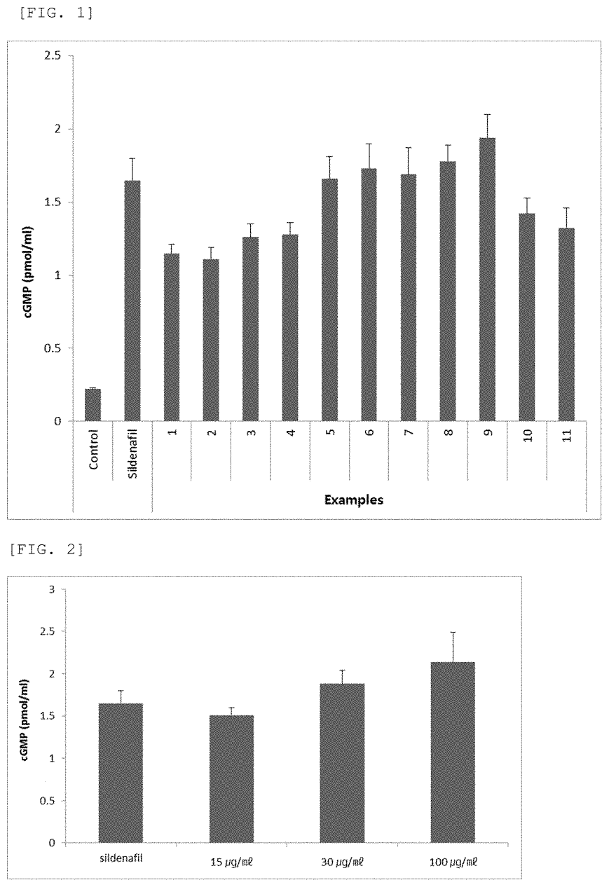

In order to examine the effects of the extracts of Examples 1-11 on cGMP in cells, each composition was adjusted to 50 μg/ml. The physiological saline was used as the negative control group and sildenafil citrate in the amount of 50 μg/ml was used as a positive control group. The results are shown in FIG. 1.

Referring to the results shown in FIG. 1, all of Examples 1-11 showed a significant increase (*p<0.05) as compared with the control group treated with saline. Also, Examples 1-5 showed a lower increase as compared to the positive control group and the composition of Example 9 showed the highest increase.

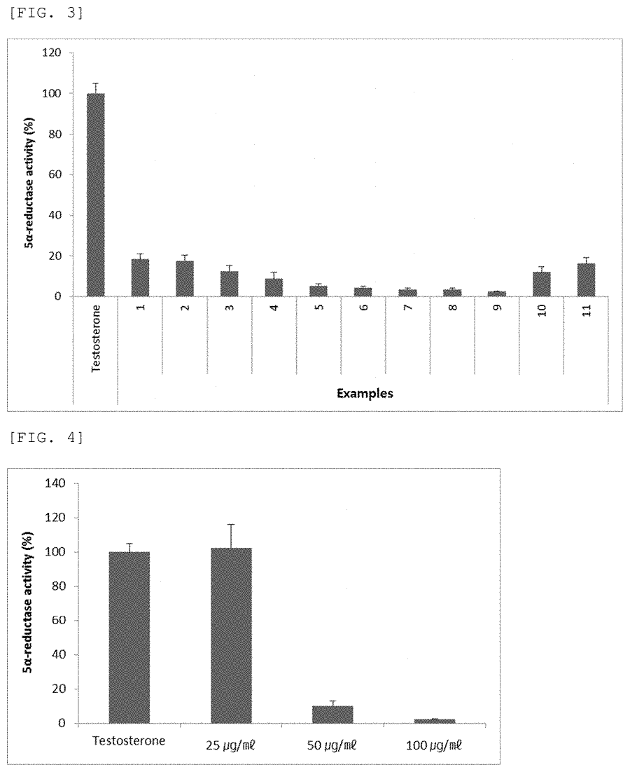

Therefore, the cells were treated with the composition of Example 9 by concentration (15, 30, 100 μg/ml), which showed excellent efficacy, and the effect on cGMP in cells was examined. As a result, as shown in FIG. 2, the test substance showed a significant increase as compared with the control group treated with physiological saline. Example 9 exhibited a tendency to increase in a concentration-dependent manner and showed the higher tendency of cGMP at the higher concentration than that of sildenafil citrate which is the positive control substance, but no statistical significance was observed

This means that the compositions of Examples 1 to 11 of the present invention have a possibility to inhibit PDE-5 expression that affects the concentration of intracellular cGMP, thereby promoting relaxation of smooth muscle cells of the corpus cavernosum penis or inhibiting contraction thereof.

### Experimental Example 2: Inhibition Experiment of Prostate Gland Hypertrophy In Vitro

The experiment was conducted using the method described in the literature to confirm that the compositions of Examples 1 to 11 of the present invention exhibit the activity in inhibiting prostatic hyperplasia in vitro (Pilar P, 2010, Adv Ther, 27(8):555-563). In order to determine the inhibitory effect on hyperactivity in prostate gland hypertrophy of 5-alpha reductase II, the prostate glands of 12 weeks old Sprague-Dawley (SD) rats were excised and tested, the phosphate buffer solution was added to the prostate glands excised from the rats and then maintained and homogenized at 4° C. to prepare a homogeneous solution, and the homogeneous solution was centrifuged at 5,000 rpm for 5 minutes to obtain a supernatant, which was used as an enzyme source. The inhibitory activity of 5-alpha reductase II was measured by ELISA assay using the compositions of Examples 1 to 11 of the present invention at a concentration of 100 μg/ml.

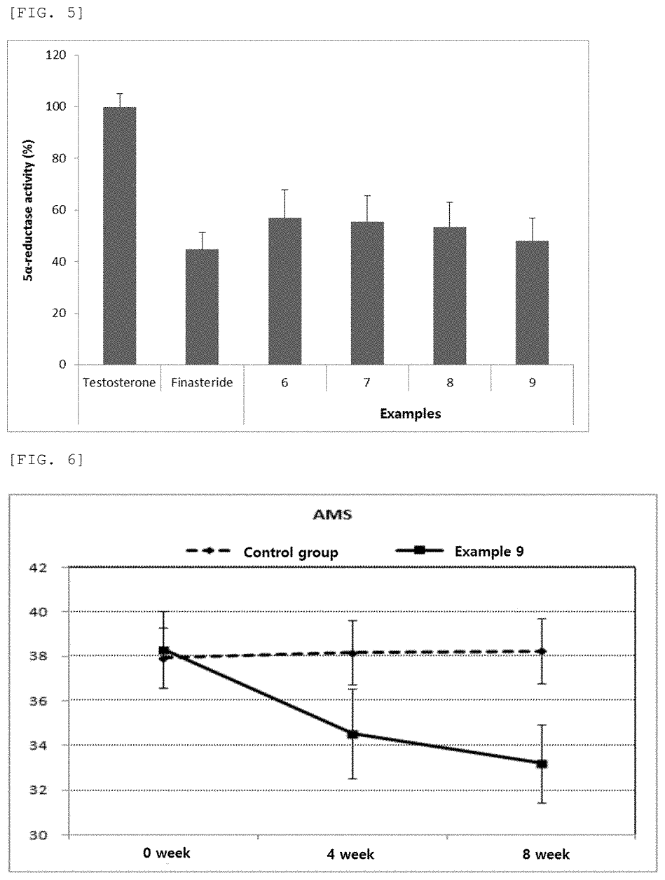

As a result of the above experiment, the activity of 5-alpha reductase by the compositions of Examples 1 to 11 of the present invention is shown in FIG. 3. This activity was expressed as the relative value of the group treated with each example, as compared with the value of the group treated with testosterone which is expressed as 100%, and the lower the value, the more excellent the inhibitory activity. All of the compositions of Examples 1 to 11 of the present invention showed excellent inhibitory activity, and the composition of Example 9 showed the most excellent inhibitory activity. When the composition of Example 9 was selected and tested at concentrations of 0, 25, 50, and 100 μg/ml, the inhibitory activity was shown from at least 50 μg/ml of concentration. These results are shown in FIG. 4.

### Experimental Example 3: Measurement of Erectile Promoting Effect In Vive

Based on the above results, the effects on erectile promotion of the compositions of Examples 6 to 9 of the present invention in vivo were measured. At this time, sildenafil citrate was used as a positive control group.

Specifically, as experimental animals, six male SPF New Zealand albino rabbits (about 3.5 to 4.0 kg body weight, Samtako, Korea) per test group were used, and these rabbits were placed in cages and fed a constant amount of conventional solid feed for rabbits and water under the same condition for 4 days or more. The rabbit was fasted for 48 hours or more and then used for the test. When fasting, the rabbits were allowed to drink water freely.

To the rabbit prepared as described above, the compositions of Examples 6 to 9 of the present invention were orally administered at a dose of 50 mg/kg per animal. Meanwhile, sildenafil citrate, a positive control group, was orally administered at a dose of 20 mg/kg. After administration as described above, the erection start time and duration were measured.

After administration of the compositions of Examples 6 to 9 of the present invention as described above, as a result of measuring the erection start time and duration was measured. As a result, when the composition of Example 5-9 of the present invention was administered, the erection start time was at about 6 minutes as shown in table 2 below, and when the positive control group was administered, the erections started at about 6 minutes, thereby confirming that all of the compositions of Examples 6 to 9 of the present invention had similar erection start times as compared with the positive control group. In addition, both the compositions of Examples 6 to 9 of the present invention and the positive control group showed a maximum erection at about 19 minutes

Even in the maintenance period of the erected state is maintained, for the positive control group, no erection was observed at a time point after 120 minutes, and in the case of the composition of Example 9 among the examples of the present invention, the longest sustained erection state of 95 minutes was maintained.

TABLE 2
Erection start time and duration period

| | Examples | | | | |
|---|---|---|---|---|---|
| Category | 6 | 7 | 8 | 9 | Control group |
| Erection start time | 6.3 ± 1.2 Min. | 6.5 ± 1.4 Min. | 6.2 ± 1.5 Min. | 6.3 ± 1.3 Min. | 6.2 ± 1.1 Min. |
| Maximum Erection time | 19 ± 1 Min. | 19 ± 1 Min. | 19 ± 1 Min. | 19 ± 1 Min. | 19 ± 1 Min. |
| Erection maintenance time | 76 ± 9 Min. | 79 ± 6 Min. | 87 ± 7 Min. | 95 ± 5 Min. | 120 ± 5 Min. |

Through these results, the compositions of Examples 6 to 9 of the present invention for albino rabbits showed the erection start time and the maximum erection time similar to those of the positive control group, as compared with the positive control group, sildenafil citrate salt, and the erectile maintenance time was lower than that of the positive control group.

Accordingly, the composition of the present invention was found to have an effect on the improvement of erectile function by promoting the erection of the penis in the rabbit administered orally.

### Experimental Example 4: Inhibition Experiment of Prostatic Hyperplasia In Vivo

Based on the results of experimental example 2, the activity against prostatic hyperplasia in vivo was examined using the compositions of Examples 6 to 9 of the present invention. At this time, finasteride was used as the positive control group.

To the subjects of male SD rats of n=6 animals per group except for the control group without testosterone administration, testosterone was administered by subcutaneous injection at a dose of 3 mg/kg/day for 2 weeks to induce prostate gland hypertrophy and after 2 weeks, it was confirmed by excision that prostate gland hypertrophy was sufficiently induced. Thereafter, while continuously injecting testosterone to maintain prostate gland hypertrophy, the compositions of Examples 6 to 9 were orally administered in an amount of 50 mg/kg and the finasteride was orally administered in an amount of 2 mg/kg. After 3 weeks of drug administration, the sizes of the prostate glands was measured and the result of 5-alpha reductase II activity in the prostate gland tissue was observed

On the day of the last administration, the animals were weighed and then sacrificed, and blood was collected and the prostate glands were excised. The excised prostate glands were weighed and the weight ratio of prostate gland per body weight (prostate gland weight (g)/body weight (g)×100) was calculated and the results are shown in Table 3.

TABLE 3
Weight ratio of prostate gland per body weight

| | Non- | Testosterone treatment | | | | | |
|---|---|---|---|---|---|---|---|
| | Testosterone | Non- | | | | | |
| Items | treatment | administration | Example 6 | Example 7 | Example 8 | Example 9 | Finasteride |
| Prostate gland | 0.59 ± 0.07 | 1.34 ± 0.13 | 1.16 ± 0.11 | 1.17 ± 0.11 | 1.13 ± 0.08 | 1.02 ± 0.09 | 0.92 ± 0.11 |
| weight | | | | | | | |
| Prostate gland/ | 0.13 ± 0.01 | 0.32 ± 0.02 | 0.28 ± 0.02 | 0.27 ± 0.02 | 0.26 ± 0.02 | 0.24 ± 0.02 | 0.24 ± 0.02 |
| body weight (%) | | | | | | | |

As a result of measuring the weight ratio of prostate gland per body weight, In all of the compositions of Examples 6 to 9, there was an effect that the size of the prostate glands was reduced, and their weight in terms of the body weight was also reduced. Compared with the positive control group, examples 8 and 9 were similar without statistically significant difference.

Also, in order to measure the inhibitory effect of prostate gland hypertrophy in rats that induced prostate gland hypertrophy, the phosphate buffer solution was added to excised prostate glands, and then maintained at 4° C. to prepare a homogeneous solution, and the homogeneous solution was centrifuged at 5,000 rpm for 5 minutes to obtain a supernatant, which was used as an enzyme source. The inhibitory activity of 5-alpha reductase II was measured by ELISA assay. When the activity of 5-alpha reductase II enzyme in rats who induce prostate gland hypertrophy by testosterone is expressed as 100%, all of the positive control group, i.e., pinasteride administration group, and examples 6 to 9 showed a corresponding inhibitory effect on the prostate gland hypertrophy, and the results are as shown in FIG. 5.

### Experimental Example 5: Human Experiment

Based on the results of Experimental Examples 4 and 5, capsules containing the composition of Example 9 of the present invention in an amount of 400 mg respectively was prepared and also capsules as a control drug were prepared using starch, and then Aging Males' Symptoms and erectile function in human subjects were assessed by ingesting one capsule per day.

The mean age of subjects participated in the human body application test is shown in Table 4. The Example 9 application group (59.1±7.62 years old) was slightly higher than the control group (57.02±8.38 years), but it was not statistically significant (p=0.214). There was no significant difference in height (p=0.092), body weight (p=0.648) and BMI (p=0.146) between the Example 9 application group and the control group, and thus it was confirmed that there was no prejudice among the subjects by the initial random assignment.

TABLE 4
Mean age of subjects (unit: the number of person (%),
mean ± standard deviation (minimum value-maximum value))

| | Control group | Example 9 | |
|---|---|---|---|
| Items | (N = 44) | (N = 44) | p |
| Age | 57.02 ± 8.38 | 59.16 ± 7.62 | 0.214 |
| | (41-70) | (41-70) | |
| Height (cm) | 170.09 ± 4.68 | 168.17 ± 5.85 | 0.092 |
| | (161.5-181.1) | (158.1-182.4) | |
| Body weight (kg) | 70.22 ± 7.36 | 71.10 ± 10.32 | 0.648 |
| | (60.3-90.2) | (50.1-96.5) | |
| BMI (kg/m²) | 24.26 ± 2.21 | 25.12 ± 3.18 | 0.146 |
| | (21.0-28.8) | (18.7-33.1) | |

Table 5 shows the results of checking over the medical history (including surgical operation) and the treatment history at the time of selection of the human application test of the subjects. As a result, it was found that both the control group and the Example 9 application group had no medical history and treatment history.

If there is a subject with medical history and treatment history who has a drug to be co-administered together with the health functional food for the present human application test, in order to assess whether the drug can be co-administered, the presence or absence of the drug to be co-administered was checked over. As a result, both the control group and the Example 9 application group showed that there were no concomitant drugs to be co-administered to treat the current medical history, thereby confirming that there was no problem on the progress of the human body application test.

TABLE 5
Check results of concomitant drug (unit: the number of person (%))

| | Control group | Example 9 | Total | |
|---|---|---|---|---|
| Items | (N = 44) | (N = 44) | (N = 88) | p* |
| Medical & treatment | | | | |
| History | | | | |
| Absence | 44(100.0) | 44(100.0) | 88(100.0) | — |
| Existence | 0(0.0) | 0(0.0) | 0(0.0) | |
| Concomitant dug for | | | | |
| existence of | | | | |
| Medical & treatment | | | | |
| History | | | | |
| Absence | 0(0.0) | 0(0.0) | 0(0.0) | — |
| Existence | 0(0.0) | 0(0.0) | 0(0.0) | |

p*: Fisher's exact test.

<Measurement for the Presence or Absence of Improvement in Aging Males' Symptoms>

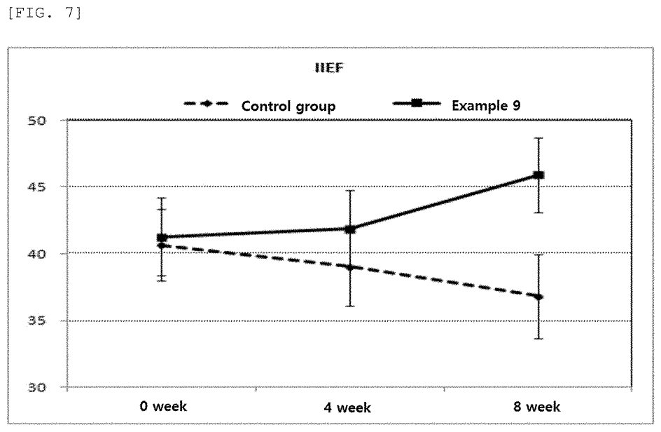

As a primary efficacy evaluation for Example 9 of the present invention, the Aging Males' Symptoms (AMS) questionnaire from subjects were comparatively evaluated, and the results are shown in Table 6 and FIG. 6. The Aging Males' Symptoms (AMS) questionaire has a total of 17 items and all items were quantified as no symptoms (1 point), lightness (2 points), moderate (3 points), severe (4 points). The total score of 27 points or more is judged to be positive. According to the severity of symptoms, 17 to 26 points are classified as no symptoms, 27 to 36 points as mild, 35 to 49 points as moderate, and 50 points or more as severe.

For the AMS total score, there was statistically significant changes in the Example 9 application group (p<0.001) according to the visit time. Also, for the comparison between the control group and the application group according to the visit time, there was a statistically significant difference at the 8th week (p=0.029). The AMS total score at 8th week after food administration in the control group was increased, whereas the degree of climacteric symptoms in the Example 9 application group was improved from moderate to mild.

TABLE 6
Evaluation of the improvement of Aging Males' Symptoms

| | | Control group | | Example 9 | | |
|---|---|---|---|---|---|---|
| | | (N = 44) | | (N = 44) | | |
| | | Mean ± S.D | | Mean ± S.D | | |
| Items | Visit | (Min-Max) | p | (Min-Max) | p | p* |
| AMS | 0 | 37.93 ± 8.9 | 0.699 | 38.30 ± 11.3 | <0.001 | 0.868 |
| (Total score) | week | (27-60) | | (27-65) | | |
| | 4 | 38.16 ± 9.5 | | 34.52 ± 13.2 | | 0.144 |
| | week | (27-64) | | (17-61) | | |
| | 8 | 38.23 ± 9.6 | | 33.20 ± 11.5 | | 0.029 |
| | week | (27-64) | | (17-57) | | |

p: Repeated measure ANOVA(according to visit time),
p*: Independence sample t-test(control group and Example 9 application group).

Table 7 shows the results of analyzing the change amounts in the Aging Males' Symptoms (AMS) questionnaire scores of the subjects. As a result, in the case of the AMS total score when comparing with baseline symptom severity, the control group showed an increase of 0.30 points, whereas the Example 9 application group showed a decrease of 5.09 points, thereby showing a statistically significant improvement (p=0.001). Especially, in the case of the Example 9 application group, it was confirmed that the degree of climacteric symptoms improved from moderate to mild.

As a result of comparing the improvement rates of the Aging Males' Symptoms (AMS) questionnaire scores from the subjects, the control group showed an improvement of 11 persons (25.0%), whereas the Example 9 application group showed an improvement of 31 persons (70.5%), thereby showing a statistically significant difference (p<0.001), and it was confirmed that the Example 9 application group helps to improve climacteric symptoms.

TABLE 7
Measurement of the improvement rates in Aging
Males' Symptoms (AMS) questionnaire scores

| | Control group | Example 9 | |
|---|---|---|---|
| Category | (N = 44) | (N = 44) | p* |
| Improvement | 11(25.0%) | 31(70.5%) | <0.001 |
| Non-Improvement | 33(75.0%) | 13(29.5%) | |

p*: chi-square test.

<Evaluation of the International Index of Erectile Function>

The questionnaire score of the international index of erectile function (IIEF) from the subjects was compared and evaluated for Example 9 of the present invention and shown in Table 8 and FIG. 7. The international index of erectile function (IIEF) has a total of 15 items and all items were quantified from 0 to 5 or 1 to 5 points.

TABLE 8
Results of evaluation of the international index of erectile function

| | | Control group | | Example 9 | | |
|---|---|---|---|---|---|---|
| | | (N = 44) | | (N = 44) | | |
| | | Mean ± S.D | | Mean ± S.D | | |
| Items | Visit | (Min-Max) | p | (Min-Max) | p | p* |
| IIEF | 0 | 40.66 ± 17.9 | 0.008 | 41.27 ± 19.2 | 0.015 | 0.877 |
| (Total score) | week | (5-72) | | (5-69) | | |
| | 4 | 39.07 ± 19.4 | | 41.91 ± 19.0 | | 0.490 |
| | week | (5-73) | | (5-72) | | |
| | 8 | 36.84 ± 20.8 | | 45.93 ± 18.5 | | 0.034 |
| | week | (5-73) | | (5-71) | | |

p: Repeated measure ANOVA(according to visit time),
p*: independence sample t-test(control group and Example 9 application group).

Table 9 shows the results of evaluating the change amounts of the questionnaire scores of the international index of erectile function (IIEF). As a result, for the IIEF total score, the control group showed a mean decrease of 3.82 points when comparing to baseline symptom severity, whereas the Example 9 application group showed an average increase of 4.66 points, thereby showing a significant improvement effect (p<0.001). For the Example 9 application group, it has been shown to help improve the severity of male erectile function symptoms.

As a result of comparing the improvement rates of the questionnaire scores of the international index of erectile function (IIEF) from the subjects, the control group showed an improvement of 9 persons (20.5%), whereas the Example 9 application group showed an improvement of 30 persons (68.2%), thereby showing a statistically significant difference (p<0.001) between two groups, and it was confirmed that the Example 9 application group helps to improve erectile dysfunction.

TABLE 9
Results of evaluating improvement rates of the questionnaire
scores of the international index of erectile function

| | Control group | Example 9 | |
|---|---|---|---|
| Category | (N = 44) | (N = 44) | p* |
| Improvement | 9(20.5%) | 30(68.2%) | <0.001 |
| Non-Improvement | 35(79.5%) | 14(31.8%) | |

p*: chi-square test.

1. A method for prevention or treatment of Prostatic hyperplasia comprising administering to a subject in need thereof a pharmaceutical composition comprising a Lespedeza cuneata extract.

2. The method of claim 1, wherein the pharmaceutical composition comprises a complex composition produced by mixing the Lespedeza cuneata extract and a Trigonellae semen extract.

3. The method of claim 2, wherein the complex composition contains as an effective component a mixture of the Lespedeza cuneata extract and the Trigonellae semen extract in the weight ratio of 1:1˜9.

4. The method of claim 1, wherein the Lespedeza cuneata extract and the Trigonellae semen extract are prepared by a process comprising,
a) crushing up Lespedeza cuneata and Trigonellae semen respectively;
b) extracting each of the above crushed materials with water, C1-C6 lower alcohol, a mixed solvent of water and C1-C6 lower alcohol, or C1-C6 organic solvent;
c) filtering the obtained extract and removing the solvent;
d) concentrating and sterilizing the filtered extract; and
e) drying and pulverizing the sterilized extract.

5. The method of claim 4, wherein step b) is performed by adding aqueous solution of 50 to 90% ethanol in the amount of 3 to 15 times the volume of the pulverized product to each of the pulverized products and then heating and extracting at 50 to 100° C. for 2 to 6 hours.

6. The method of claim 4, wherein step b) is repeated 1 to 7 times.

7. The method of claim 2, wherein the content of the complex composition comprising the Lespedeza cuneata extract and the Trigonellae semen extract is 0.01 to 50 mg/kg.

8. (canceled)

9. A method for improvement or treatment of erectile function comprising administering to a subject in need thereof a pharmaceutical composition comprising complex composition produced by mixing Lespedeza cuneata extract and Trigonellae semen extract.

10. A method for improvement of erectile function and for prevention or treatment of prostatic hyperplasia at the same time among Aging Males' Symptoms comprising administering to a subject in need thereof a pharmaceutical composition comprising a complex composition produced by mixing Lespedeza cuneata extract and Trigonellae semen extract.
